(19) United States
(12) Patent Application Publication
(10) Pub. No.: US 2007/0259831 A1
(43) Pub. Date: Nov. 8, 2007

(54) Treatment and screening methods for promoting neurogenesis

(76) Inventor: William A. Carlezon JR., Lincoln, MA (US)

Correspondence Address: CLARK & ELBING LLP, 101 FEDERAL STREET, BOSTON, MA, 02110, US

(21) Appl. No.: 11/796,651

(22) Filed: Apr. 27, 2007

Related U.S. Application Data
(60) Provisional application No. 60/795,397, filed on Apr. 27, 2006.

(51) Int. Cl. A61K 48/00; A61K 39/395

(52) U.S. Cl. 514/44; 424/145.1

(57) ABSTRACT

The invention features methods for promoting neurogenesis including treatment of psychiatric disorders (e.g., depression, bipolar disorder, and post traumatic stress disorder), drug abuse or addiction, neurodegenerative diseases (e.g., Alzheimer's disease, Parkinson's disease, amyotrophic lateral sclerosis, multiple sclerosis, frontotemporal dementia, Huntington's disease, and prion disease), and head trauma (e.g., stroke and physical injury) by inhibition of Sprouty (SPRY) and methods for identification of compounds useful promoting neurogenesis (e.g., useful in the treatment of psychiatric disorders, drug abuse or addiction, neurodegenerative diseases, or head trauma).

### CROSS-REFERENCE TO RELATED APPLICATIONS

This application claims benefit of U.S. Provisional Application No. 60/795,397, filed Apr. 27, 2006, which is hereby incorporated by reference.

### BACKGROUND OF THE INVENTION

The invention relates to treatment and screening methods for promoting neurogenesis.

Psychiatric disorders, including depression, bipolar disorder, and post traumatic stress disorder, and neurodegenerative diseases, including Alzheimer's disease, Parkinson's disease, amyotrophic lateral sclerosis, multiple sclerosis, frontotemporal dementia, Huntington's disease, and prion disease, as well as drug abuse or addiction and head trauma, such as stroke or physical injury, affect millions worldwide each year. Treatments of such psychiatric disorders and addictions often only exhibit limited effectiveness and no treatments are known which can reverse the progression of neurodegenerative disease.

Previous work has indicated that neuronal loss is a common thread in psychiatric disorders, drug abuse or addiction, and neurodegenerative diseases. Neuronal loss may be due to a decrease in neuronal proliferation or an increase in neuronal death. The causes of the neuronal loss in these disorders and diseases are not well understood.

The Sprouty family of proteins was initially identified as a negative regulator of receptor tyrosine kinases and cellular proliferation in development of Drosophila. Four mammalian Sprouty homologues (SPRY 1-4) have been identified. These likewise have been implicated in negative regulation of receptor tyrosine kinases and cellular proliferation during development.

### SUMMARY OF THE INVENTION

The invention features methods for promoting neurogenesis in a subject and methods for identifying compounds useful in promoting neurogenesis.

In a first aspect, the invention features a method for promoting neurogenesis in a subject (e.g., a human, a domesticated animal, or a laboratory animal) which includes administering to the subject a composition that selectively inhibits Sprouty (e.g., human Sprouty, such as Sprouty1, Sprouty2, Sprouty3, or Sprouty4) to a degree sufficient to promote neurogenesis, particularly in spinal cord or the brain (e.g., the hippocampus or prefrontal cortex). The composition may include a compound that specifically binds Sprouty (e.g., an antibody that specifically binds Sprouty or a Sprouty-binding fragment thereof). In another embodiment, the composition may include a dominant negative form of Sprouty (e.g., a dominant negative Sprouty fragment) such as a Sprouty protein including a mutation at a tyrosine (e.g., position 53 of human Sprouty1, position 55 of human Sprouty2, position 27 of human Sprouty3, or position 52 of human Sprouty4). The mutation at a tyrosine may be a tyrosine-to-alanine point mutation or a tyrosine-to-phenylalanine point mutation. In a particular embodiment, the mutation is a Y55F mutation in Sprouty2.

In other embodiments, the composition may include an siRNA molecule that specifically binds to an mRNA encoding Sprouty, a vector encoding an siRNA that specifically binds to a mRNA encoding Sprouty, or a vector encoding a dominant negative form of Sprouty (e.g., a vector encoding any dominant negative Sprouty protein described above such as Y55F Sprouty2).

In a second aspect, the invention features a method for promoting neurogenesis in a subject (e.g., a human such as an adult human, a domesticated animal, a laboratory animal). The method includes administering to the subject a composition that inhibits Sprouty activity (e.g., human Sprouty, such as Sprouty1, Sprouty2, Sprouty3, or Sprouty4) by binding to a Sprouty binding site to a degree sufficient to promote neurogenesis. The composition may include a dominant negative form of Sprouty (e.g., a dominant negative Sprouty fragment) such as a Sprouty protein including a mutation at a tyrosine (e.g., position 53 of human Sprouty1, position 55 of human Sprouty2, position 27 of human Sprouty3, or position 52 of human Sprouty4). The mutation at a tyrosine may be a tyrosine-to-alanine point mutation or a tyrosine-to-phenylalanine point mutation. In a particular embodiment, the mutation is a Y55F mutation in Sprouty2. In certain embodiments, the Sprouty binding site is on a protein that binds Sprouty (e.g., GRB2, c-CBL, GAP1, Caveolin-1, CIN85, Cbl-b, B-Raf, Raf1, FRS2, Shp2, PTP1B, or TESK1). In some embodiments, the subject is a human, rat, mouse, dog, or chimpanzee.

In either of the above two aspects, the subject, prior to being treated, may be diagnosed with a psychiatric disorder (e.g., depression, bipolar disorder, or post traumatic stress disorder) where the promoting of neurogenesis treats the psychiatric disorder. In another embodiment, the subject, prior to being treated, may be diagnosed with a have a neurodegenerative disease (e.g., Alzheimer's disease, Parkinson's disease, amyotrophic lateral sclerosis, multiple sclerosis, frontotemporal dementia, Huntington's disease, or prion disease) where the promoting of neurogenesis treats the neurodegenerative disease. In yet another embodiment, the subject abuses or is addicted to a drug, where the promoting of neurogenesis decreases use of the drug or treats the addiction. In other embodiments, the subject has suffered head trauma (e.g., physical injury or stroke), where the promoting of neurogenesis increases the rate or extent of recovery from the trauma. In embodiments where the subject suffers from a psychiatric disorder such as depression, bipolar disorder, or post traumatic stress disorder, the method may further include administration of an additional antidepressant therapy such as a chemical antidepressant (e.g., those described herein) or electroconvulsive therapy (ECT). In other embodiments, administration may take peripherally (e.g., to promote peripheral neurogenesis).

The invention, in a third aspect, features a method for identifying a candidate compound useful in promoting neurogenesis which including the steps (a) contacting a compound with a Sprouty protein (e.g., human Sprouty protein such as Sprouty1, Sprouty2, Sprouty3, or Sprouty4); and (b) measuring the binding of the compound to the Sprouty protein, where specific binding of the compound to the Sprouty protein identifies the compound as a candidate compound useful in promoting neurogenesis in a subject. The method may further include step (c) administering to a non-human mammal (e.g., a rodent such as a rat or mouse) a compound identified in step (b) as specifically binding Sprouty, where a compound that increases neurogenesis in the mammal is a identified a potential therapeutic compound. The mammal may have at least one symptom of a psychiatric disorder or neurodegenerative disease or may have a psychiatric disorder or a neurodegenerative disease or have suffered from a head trauma.

In a fourth aspect, the invention features a method for identifying a candidate compound useful in promoting neurogenesis in a subject which includes the steps (a) contacting a compound (e.g., a compound from a chemical library) with a cell or cell extract which includes a polynucleotide encoding a Sprouty protein such as a human Sprouty protein (e.g., Sprouty1, Sprouty2, Sprouty3, or Sprouty4); and (b) measuring the level of Sprouty expression in the cell or cell extract, where a decreased level of Sprouty expression in the presence of the compound relative to the level in the absence of the compound identifies the compound as a candidate compound useful in promoting neurogenesis in a subject. The method may further include step (c) administering to a non-human mammal (e.g., a rodent such as a rat or mouse) a compound identified in step (b) as reducing Sprouty expression, where a compound that increases neurogenesis in the mammal is a identified a potential therapeutic compound. The mammal may have at least one symptom of a psychiatric disorder or neurodegenerative disease or may have a psychiatric disorder or a neurodegenerative disease or have suffered from a head trauma.

In a fifth aspect, the invention features a method for identifying a candidate compound useful in promoting neurogenesis in a subject which includes the steps (a) contacting a compound (e.g., a compound from a chemical library) with a Sprouty target protein (e.g., GRB2, c-CBL, GAP1, Caveolin-1, CIN85, Cbl-b, B-Raf, Raf1, FRS2, Shp2, PTP1B, or TESK1); and (b) measuring the binding of the compound to the Sprouty-target protein, where specific binding of the compound to the Sprouty-target protein identifies the compound as a candidate compound useful in promoting neurogenesis in a subject. The method may further include a step determining whether said compound decreases binding of Sprouty to the target protein or may further include a step administering to a non-human mammal (e.g., a laboratory animal such as a rat or mouse) a compound identified as specifically binding Sprouty-target protein, where a compound that increases neurogenesis in the mammal is a identified a potential therapeutic compound. The mammal may have at least one symptom of a psychiatric disorder or neurodegenerative disease or may have a psychiatric disorder or a neurodegenerative disease or have suffered from a head trauma.

In any of the above aspects, the psychiatric disorder may be depression, bipolar disorder, or post traumatic stress disorder, or the neurodegenerative disease may be Alzheimer's disease, Parkinson's disease, amyotrophic lateral sclerosis, multiple sclerosis, frontotemporal dementia, Huntington's disease, or prion disease. The head trauma may be a stroke or physical injury. Further, in any of the above aspects, the methods may employ a Sprouty protein from any organism (e.g., fly, chicken, or a mammal such as a mouse, rat, dog, chimpanzee, or human) and may be any Sprouty variant from the organism (e.g., human Sprouty1, human Sprouty2, human Sprouty3, or human Sprouty4). Further, in any of the above aspects, fragments of Sprouty (e.g., C-terminal fragments containing the cysteine-rich domain or N-terminal fragments containing a conserved tyrosine) may be used in place of the full length Sprouty protein.

By “Sprouty” or “Sprouty protein” is meant a protein having at least 50%, 60%, 70%, 80%, 90%, 95%, 99%, or even 100% identity to any of SEQ ID NOS:1-5.

By “Sprouty fragment” is meant a portion of the full length Sprouty protein that is at least 4, 5, 10, 15, 20, 30, 40, 50, 100, or 200 amino acids in length. Sprouty fragments may include conserved regions (e.g., the amino-terminal region or carboxy-terminal region) of Sprouty. Sprouty fragments may be used in any of the treatment or screening methods of the invention in place of the full length protein. In certain embodiments, Sprouty fragments are capable of inhibiting cellular proliferation. Alternatively, Sprouty fragments can act as dominant negative proteins (i.e., can increase cellular proliferation). Determination of the effect of a Sprouty fragment on cellular proliferation may be performed using any method known in the art (e.g., those described herein).

By “subject” is meant either a human or non-human animal (e.g., a mammal such as a primate). Subjects include domesticated animals, laboratory animals, farm animals, and zoo animals. Exemplary non-human animals include dogs, cats, rats, mice, horses, cows, sheep, chimpanzees, and monkeys. A subject may be an adult (e.g., a human adult older than 18, 25, 30, 40, 50, 60, or 70 years), may be a juvenile (e.g., a human between ages one and eighteen years, or may be an infant (e.g., a human less than one year old).

By a composition that “promotes neurogenesis” is meant a composition that increases proliferation of neurons by at least 1%, 2%, 5%, 10%, 25%, 50%, 100%, 200%, 500%, 1000%, 10,000%, or 100,000% as compared to in the absence of the composition.

By “Sprouty binding site” is meant the portion of any molecule to which a Sprouty protein binds under physiological conditions. Exemplary molecules to which Sprouty binds include GRB2, c-CBL, GAP1, Caveolin-1, CIN85, Cbl-b, B-Raf, Raf1, FRS2, Shp2, PTP1B, and TESK1. Sprouty itself may include a Sprouty binding site (e.g., hetero or homo-oligomerization), as well as sites on the plasma membrane.

A composition that “selectively inhibits Sprouty” means a composition that (i) includes a compound that binds specifically to the Sprouty protein (e.g., a small molecule or a Sprouty antibody), specifically binds the Sprouty mRNA (e.g., a siRNA molecule), decreases expression of the gene encoding Sprouty, or prevents the Sprouty protein from performing its normal function (e.g., prevents binding of Sprouty to a target molecule such as growth-factor-receptor substrate-2 (GRB2), RAF, or GAP1 or prevents binding of Sprouty to the plasma membrane), and (ii) is capable of increasing cellular proliferation upon administration to a subject.

“Treating” a disease or condition in a subject or “treating” a subject having a disease or condition refers to subjecting the individual to a treatment, e.g., a pharmaceutical treatment such as the administration of a drug, such that at least one symptom of the disease or condition is decreased, stabilized, or prevented.

By “specifically binds” or “specific binding” is meant a compound or antibody which recognizes and binds a polypeptide (e.g., a Sprouty protein or a Sprouty target molecule) but which does not substantially recognize and bind other molecules in a sample, for example, a biological sample, which naturally includes the polypeptide.

By “decrease in the level of expression or activity” of a gene is meant a reduction in protein or nucleic acid level or activity in a cell, a cell extract, or a cell supernatant. For example, such a decrease may be due to reduced RNA stability, transcription, or translation, increased protein degradation, or RNA interference. In certain embodiments, this decrease is at least 5%, 10%, 25%, 50%, 75%, 80%, or even 90% of the level of expression or activity under control conditions.

Other features and advantages of the invention will be apparent from the following Detailed Description, the drawings, and the claims.

### BRIEF DESCRIPTION OF THE DRAWINGS

FIGS. 1A-1C are graphs showing numbers of labeled cells in the ILPFC (Mean ±SEM). White bars represent controls, black bars represent ECS-treated animals. ECS increased numbers of BrdU- (FIG. 1A) and PCNA-immunoreactive cells (FIG. 1B), but reduced numbers of SPRY-immunoreactive cells (FIG. 1C). Only total cell number estimates are shown, but cell density numbers showed exactly the same pattern. *p<0.05, **p<0.01 for ECS-treated compared to control.

FIGS. 2A and 2B are photomicrographs showing BrdU labeled cells and PCNA-immunoreactive cells. FIG. 2A shows BrdU-labeled cells distributed throughout the cortical layers within the ILPFC in representative from control (left) and ECS-treated (middle) brains. No specific staining is observed when the primary antibody is omitted from the staining protocol (right). Cortical surface is on top of the photomicrographs, white matter is at the bottom, and the scale bar is 100 μm. FIG. 2B shows a similar pattern of staining is seen for PCNA-immunoreactive cells in control (left) and ECS (middle). PCNA labeling was specific for cell nuclei (right; scale bar is 20 μm).

FIGS. 3A-3C are photomicrographs showing double- and triple-labeling experiments. FIG. 3A shows cell profiles from the same ECS brain labeled only with NG2 (left) or BrdU (center) demonstrate the features of each stain. A double-labeled cell from the same brain (right) shows a nucleus stained with BrdU and cell body and processes stained with NG2 (scale bar is 20 μm). FIG. 3B cell profiles from the same ECS brain labeled with PLP (left) or BrdU+PLP (right). FIG. 3C shows examples of cell profiles from immunofluorescence studies: double-labeling for RECA (green) and BrdU (red) (left) and triple labeling for NeuN (green), S100β (blue) and BrdU (red) (right). No double- or triple-labeled cells were found in any section.

FIGS. 4A and 4B are photomicrographs showing patterns of SPRY2 labeling. FIG. 4A shows that, throughout the ILPFC, SPRY2 immunoreactivity is stronger in control (left) than in ECS-treated brains (right). Cortical surface is on top of the photomicrographs, white matter is at the bottom, and the scale bar is 100 μm. FIG. 4B shows a higher magnification of SPRY2 labeling in a control brain (left) indicates cytoplasmic staining (scale bar is 20 μm). There is no specific staining when the primary antibody is omitted from the staining protocol (right).

FIGS. 5A-5H are photomicrographs showing hippocampus (HIP) tissue after viral-mediated gene transfer in a rat. HSV-LacZ was infused into the left HIP, and HSV-Y55F-SPRY2, described below, was infused into the right HIP; arrows indicate infusion sites. BrdU was administered 7 days after gene transfer; rats were killed 24 hr later. FIG. 5A shows BrdU-labeling after infusion of HSV-LacZ (50×). The dentate gyrus (DG) and hilus (HIL) are indicated; the black bar represents 500 μm. FIGS. 5B and 5C show higher magnification (200×) of the areas indicated by the white (FIG. 5B) and black boxes (FIG. 5C) of FIG. 5A. FIG. 5D shows BrdU-labeling after infusion of HSV-Y55F-SPRY2 (50×). FIGS. 5E and 5F show higher magnification (200×) of the areas indicated by the white (FIG. 5E) and black boxes (FIG. 5F) of FIG. 5D. FIG. 5G shows that 28 days after gene transfer, more BrdU-labeled cells survived in hemispheres treated with HSV-Y55F-SPRY2 than in hemispheres treated with HSV-LacZ (net cell #=BrdU cells on the Y55F side−BrdU cells on the LacZ side). By contrast, net cell numbers were lowest after treatment with HSV-wt-SPRY2 (encoding wild-type SPRY2). FIG. 5H is a photomicrograph showing new neurons at the 28-day time point co-labeled with BrdU and NeuN are indicated by white arrows; labeled cells out of the focal plane are indicated by gray arrows. *P<0.05, Fisher's tests.

FIG. 6 is a schematic representation showing that therapeutic inhibition of Sprouty by, for example, ECS, Y55F-SPRY, or drugs such as chemical antidepressants, leads to increased cellular proliferation of neurons. This may be useful in the treatment of psychiatric disorders, neurodegenerative diseases, drug abuse or addiction, or head trauma. Arrows indicate stimulation; flat lines indicate inhibition. Growth factors (GF) such as FGF, EGF, or VEGF act at growth factor receptors (GFR) that regulate cellular proliferation in the HIP. GF-induced activation of GFRs stimulates ERK/MAPK signaling pathways and increases transcription of SPRY, which, in turn, exerts inhibitory feedback on subsequent GF-stimulation of ERK. ECS and dominant negative (Y55F)-SPRY inhibit SPRY, thereby promoting cellular proliferation.

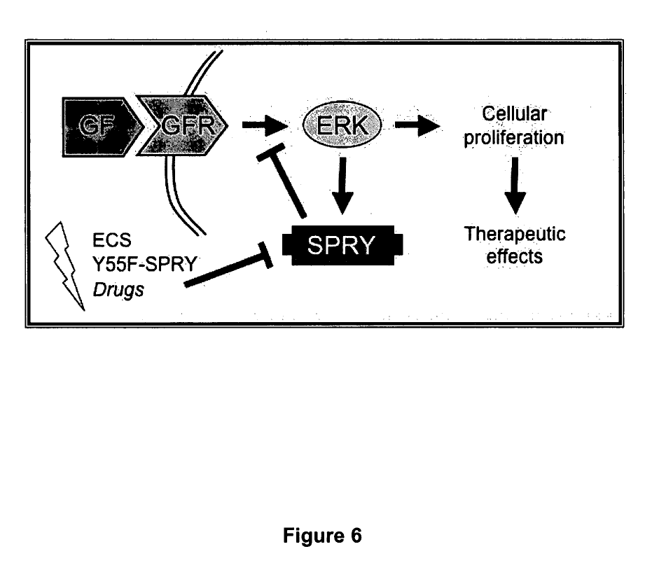

FIG. 7 contains the sequences of human Sprouty proteins (SPRY1-4; SEQ ID NOS:1-4), rat Sprouty subtype 2 (SEQ ID NO:5), dominant negative Y55F rat Sprouty subtype 2 (SEQ ID NO:6), and Y55F human Sprouty subtype 2 (SEQ ID NO:7).

FIG. 8 is a schematic diagram showing coronal sections through the rat brain (Paxinos et al., infra) with the ILPFC indicated by the stippled pattern. The ILPFC is bounded by the prelimbic prefrontal cortex dorsally and dorsal peduncular cortex ventrally.

### DETAILED DESCRIPTION

Based on the discovery of Sprouty as a negative regulator of cellular proliferation in the adult mammalian brain, the invention features methods for promoting neurogenesis in a subject such as a subject suffering from a psychiatric disorder (e.g., depression, bipolar disorder, and post traumatic stress disorder), drug abuse or addiction, a neurodegenerative disease (e.g., Alzheimer's disease, Parkinson's disease, amyotrophic lateral sclerosis, multiple sclerosis, frontotemporal dementia, Huntington's disease, and prion disease), or head trauma (e.g., stroke or physical injury) by inhibition of Sprouty (SPRY) and methods for identification of compounds useful in promoting neurogenesis (e.g., useful in the treatment of psychiatric disorders, drug abuse or addiction, neurodegenerative diseases, and head trauma) by contacting a Sprouty protein, Sprouty fragment, or Sprouty target protein (e.g., GRB2, RAF, c-CBL, GAP1, or any Sprouty target protein described herein) with a compound and determining whether the compound binds to the protein as well as methods for determining whether a compound reduces Sprouty expression in a cell containing a polynucleotide encoding Sprouty.

Cellular Proliferation and Disease

Neuronal loss, either due to increased cellular death or decreased cellular proliferation, has been identified in subjects suffering from psychiatric disorders including depression, bipolar disorder, and post traumatic stress disorder, in subjects who abuse a drug or have a drug addiction (e.g., involving a drug described herein), and in subjects with neurodegenerative diseases including Alzheimer's disease (AD), Parkinson's disease (PD), amyotrophic lateral sclerosis (ALS), multiple sclerosis (MS), frontotemporal dementia (FTD), Huntington's disease (HD), and prion disease (e.g., Creutzfeldt-Jakob disease). Neuronal loss can also occur in subjects suffering from a head trauma such as a stroke or physical injury.

Psychiatric Disorders

A link between cellular proliferation, both in the pathophysiology and in the treatment of psychiatric disorders such as depression and post traumatic stress disorder has been identified. Specifically, increased neuronal atrophy and cell loss has been observed in humans suffering from depression, as well as in animal models of depression. Further, previous work has indicated that subjects receiving electroconvulsive seizure therapy (ECS) and other antidepressive treatments exhibit upregulated expression of growth factors (Newton et al., J. Neurosci. 23:10841-10851, 2003; Malberg et al., Rev. Psychiatr. Neurosci. 29:196-204, 2004). However, mechanisms underlying these changes, prior to the invention, have not been well understood.

Drug Abuse or Addiction

There is a growing body of evidence that drug abuse or addiction decreases cell proliferation in the brain, for example, in the hippocampus (Yamaguchi et al., Synapse 58:63-71, 2005). Further, co-morbidity with a psychiatric disorder such as depression or attention deficit hyperactivity disorder (ADHD) may be involved in development or maintenance of the abuse or addiction, suggesting that decreased neurogenesis may be involved in both psychiatric disorders and drug abuse or addiction. This possibility suggests that drug abuse or addiction, either alone or in combination with a psychiatric disorder, may also be treated by promoting neurogenesis.

Drugs which can be abused or drugs to which a subject may become addicted include cannabinoids (e.g., hashish and marijuana); depressants such as barbiturates (e.g., Amytal, Nembutal, Seconal, and Phenobarbital), benzodiazepines (e.g., Ativan, Halcion, Librium, Valium, and Xanax), gamma-hydroxybutyrate, and methaqualone; dissociative anesthetics (e.g., ketamine, PCP, and PCP analogs); hallucinogens (e.g., LSD, mescaline, and psilocybin); opioids and morphine derivatives (e.g., codeine, fentanyl, fentanyl analogs, heroin, morphine, opium, oxycodone HCl, and hydrocodone bitartrate); stimulants (e.g., amphetamine, cocaine, MDMA (methylenedioxy-methamphetamine), methamphetamine, methylphenidate, and nicotine), and inhalants such as solvents (e.g., paint thinners, gasoline, and glues), gases (e.g., butane, propane, aerosol propellants, and nitrous oxide), nitrites (e.g., isoamyl, isobutyl, and cyclohexyl).

Neurodegenerative Diseases

Subjects suffering from a neurodegenerative disease, e.g., those described herein, exhibit loss of neuronal populations. For example, subjects with AD exhibit loss of cortical neurons, and subjects with PD exhibit loss of dopaminergic neurons critical for proper motor function. While causes of the observed neuronal losses in neurodegenerative diseases are not fully understood, the discovery of Sprouty as playing a role in the inhibition of neuronal growth factors in adult mammals provides a therapeutic strategy in the treatment of such neurodegenerative diseases.

Trauma

Subject suffering a head trauma, such as stroke or a physical injury, can benefit from increased neurogenesis. Head trauma can result in loss or death of neurons in the subject, and treatment with a molecule that stimulates neurogenesis by selectively inhibiting Sprouty can therefore provide a treatment strategy for such subjects. Increasing neurogenesis is such subject may increase the rate of recovery, or improve the extent of recovery from the trauma.

Sprouty

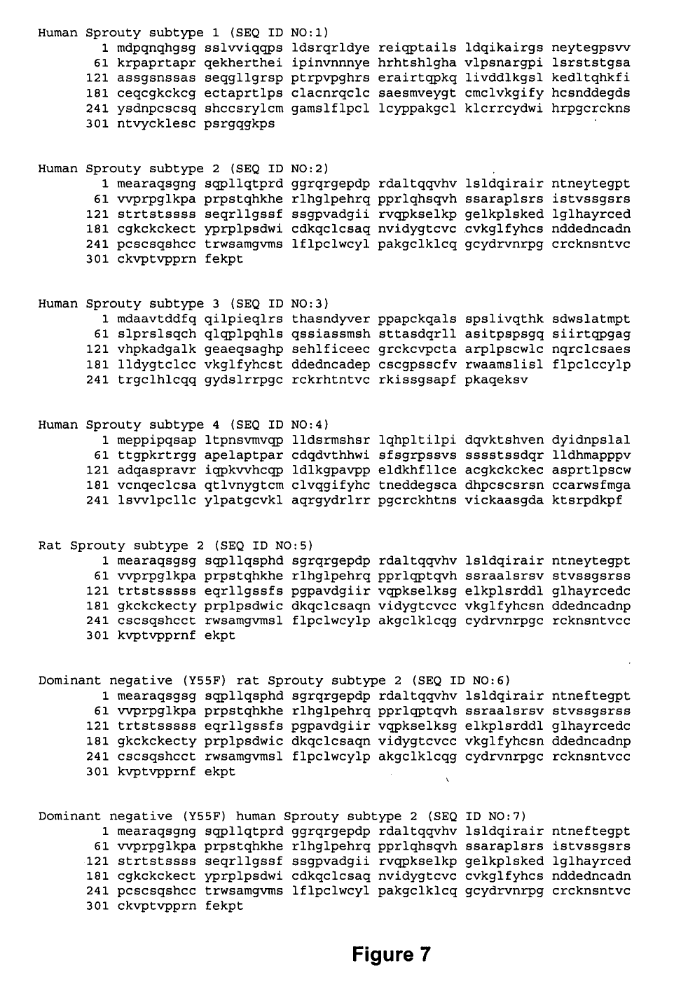

Mammalian Sprouty (SPRY) proteins (e.g., human Sprouty proteins; SEQ ID NOS:1-4; see FIG. 7) are negative regulators of certain growth factors and their associated receptor-tyrosine-kinase (RTK)-dependent signaling pathways. Sprouty proteins have a conserved amino-terminal region containing a tyrosine that undergoes phosphorylation and a conserved cysteine-rich carboxy-terminal region (residues 178-221) involved in membrane binding. These proteins inhibit cellular proliferation and migration (Impagnatiello et al., J. Cell Biol. 152:1087-1098, 2001; Yigzaw et al., J. Biol. Chem. 276:22742-22747, 2001; Kim et al. Nat. Rev. Mol. Cell. Biol. 5:441-450, 2004). Sprouty activity is dependent on localization to the plasma membrane, which may be mediated through the cysteine-rich carboxy terminal domain of Sprouty binding to phosphatidylinositol-4,5-bisphosphate in the plasma membrane. Sprouty transcription is elevated when growth factors act at RTK receptors to stimulate the extracellular signal-regulated kinase/mitogen activated protein kinase (ERK/MAPK) cascade (Kim et al. Nat. Rev. Mol. Cell. Biol. 5:441-450, 2004). In turn, Sprouty exerts feedback inhibition on this system by inhibiting the activation of ERK/MAPK signaling within multiple growth (or trophic) factor signaling pathways, including those for fibroblast growth factor (FGF), endodermal growth factor (EGF), and vascular-endothelial growth factor (VEGF) (Hacohen et al., Cell 92:253-263, 1998; Kramer et al., Development 126:2515-2255, 1999; Cabrita et al., Thromb. Haemost. 90:586-590, 2003; Rubin et al., Curr. Biol. 13:297-307, 2003; Zhang et al., Arterioscler. Thromb. Vasc. Biol. 25:533-538, 2005). Indeed, FGF2 has been implicated in the regulation of cellular proliferation processes in the adult brain (Yoshimura et al., Proc. Natl. Acad. Sci. USA 98:5874-5879, 2001; Jin et al., Aging Cell 2:175-183, 2003). This feedback mechanism enables Sprouty to provide temporal and spatial constraints upon intracellular signaling and prevents inappropriate cellular differentiation and proliferation.

The observed feedback inhibition of the ERK/MAPK signaling by Sprouty may be mediated through several interaction partners (Kim et al, Nat. Rev. Mol. Cell. Biol. 5:441-450, 2004). In particular, the SH2 domain of growth-factor-receptor substrate-2 (GRB2) binds to phosphorylated Sprouty (e.g., human SRPY2 phosphorylated at tyrosine 55). This binding prevents GRB2 from interacting with FGF-receptor substrate-2 (FRS2) or SH2-domain-containing protein tyrosine phosphatese-2 (SHP2), thereby inhibiting the ERK/MAPK signaling pathway. SPRY4 has also been observed to interact with the catalytic domain of RAF, an interaction which blocks phospholipase C-δ activation of RAF. This blockage also can decrease the activation of the ERK/MAPK kinase. Other Sprouty interaction partners include, for example, c-CBL, GAP1, Caveolin-1, CIN85, Cbl-b, B-Raf, PTP1B, and TESK1 (Mason et al., Trends. Cell. Biol. 16:45-54, 2006).

Proteins from the Sprouty family are implicated in key aspects of cellular proliferation and differentiation, angiogenesis, retinal development, trachea morphogenesis, and vascular restenosis (Gross et al., J. Biol. Chem. 276:46460-46468, 2001; Hacohen et al., Cell 92:253-263, 1998; Kim et al. Nat. Rev. Mol. Cell. Biol. 5:441-450, 2004; Lee et al., J. Biol. Chem. 276:4128-4133, 2001; Zhang et al., Arterioscler. Thromb. Vasc. Biol. 25:533-538, 2005). Although there is substantial evidence that Sprouty is involved in the development of the central nervous system (Lin et al., Genesis 41:110-115, 2005), the role of Sprouty proteins in the adult brain, prior to the present invention, had not been established.

Expression Studies of Rats Subjected to ECS

We identified a role for Sprouty function in the adult nervous system. Using rats, we discovered that a regimen of electroconvulsive seizure (ECS) that induces cellular proliferation in key brain regions (hippocampus (HIP) and prefrontal cortex) simultaneously causes decreases in the expression of SPRY2 (rat Sprouty-subtype 2; SEQ ID NO:5). As previous studies designed to catalogue ECS-induced alterations in gene expression in rats (Newton et al., J. Neurosci. 23:10841-10851, 2003) used microarrays that did not include probes for SPRY2, this result indicates, for the first time, a role for SPRY2 in adult brain and, further, led to the determination that the ECS-induced decrease in SPRY2 expression itself, as outlined below, triggers cellular proliferation.

In rats subjected to ECS treatment, there was a significant overall effect on the infralimbic prefrontal cortex (ILPFC) volume and all cell counts when analyzed together (F(1,10)=4.769, p<0.05). There were no significant differences between hemispheres (F(1,10)=0.217, ns). Thus, data from both hemispheres were averaged and the mean values were used in subsequent analyses.

Volume and Neuron/Glia Numbers

ECS had no effect on ILPFC volume. Neuronal density was slightly lower in the ECS group than in controls, whereas glial density was slightly higher; none of these differences approached significance (Table 1). Total cell numbers in the ILPFC were calculated by multiplying the density counts with ILPFC volume, and there were no differences between the two groups (Table 1).

TABLE 1
Estimated ILPFC volume and cell numbers

| | Mean | SEM | F | p Value |
|---|---|---|---|---|
| Volume (mm³) | 2.30 | .19 | .442 | .650 |
| | 2.15 | .13 | | |
| Neuron Density (cells/mm³) | 17612 | 1039 | 1.390 | .276 |
| | 16031 | 794 | | |
| Total Neurons | 40833 | 4795 | 1.262 | .308 |
| | 34493 | 2814 | | |
| Glia Density (cells/mm³) | 25858 | 1499 | .208 | .814 |
| | 26320 | 1128 | | |
| Total Glia | 59578 | 6932 | .175 | .841 |
| | 56316 | 3110 | | |

First row lists results from control group, second row from ECS-treated group.
ILPFC, infralimbic prefrontal cortex; ECS, electroconvulsive seizure.

Cell Proliferation

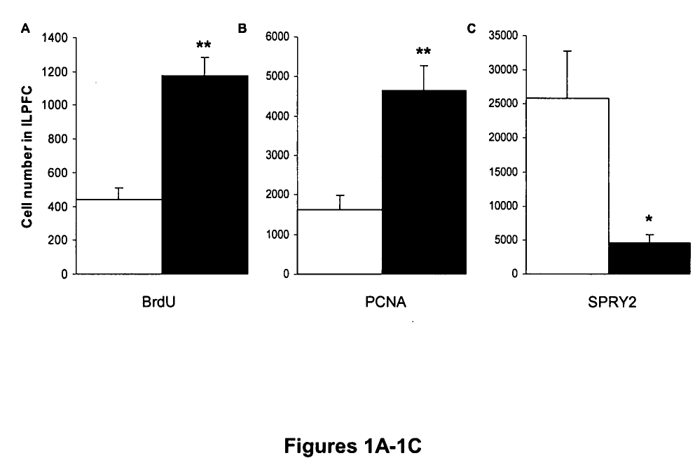

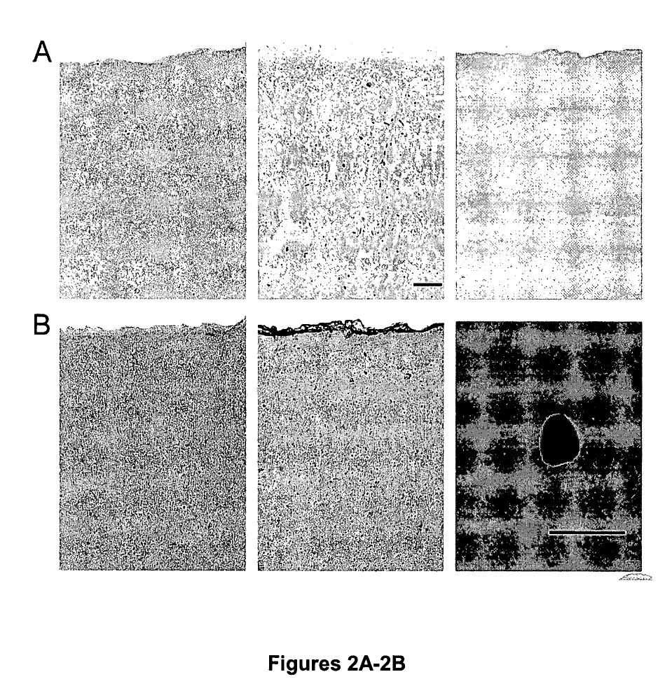

ECS caused a three-fold increase in the number of BrdU-labeled cells in the ILPFC (F(1,10)=17.374, p<0.01) (FIG. 1A). The profiles of BrdU-labeled nuclei were typically round, although they were occasionally elongated and thin (suggesting blood vessels). BrdU-labeled nuclei were evenly distributed through all cortical layers in both groups (FIG. 2A). There was negligible nonspecific staining when the primary antibody was omitted from the staining procedure (FIG. 2A). PCNA-labeled profiles were similar in morphology and location to those with BrdU (FIG. 2B). The pattern of PCNA-labeled cell numbers were also similar to that seen with BrdU, with a statistically significant three-fold increase in number of cells in the ECS group (F(1,10)=7.902, p<0.01) (FIG. 1B). For clarity, the results are presented for the total cell numbers within the ILPFC, although the density of labeled cells tracked total cell number very closely and the statistical analyses were virtually identical (data not shown).

Phenotyping

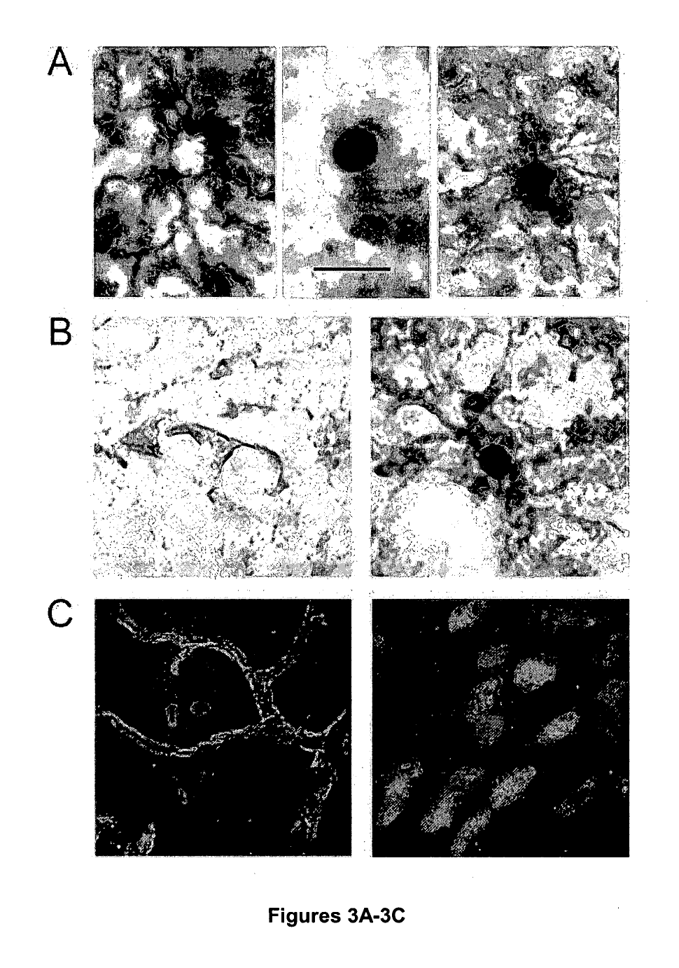

The percentage BrdU-labeled cells that were double-labeled with PLP, NG2, S100β, RECA, or NeuN in the ILPFC were then examined. NG2- and PLP-labeled cells showed a distinctive morphology with uneven deposition of label in clumps surrounding the cell body and labeled processes extending from the soma and forming a reticular pattern in the parenchyma (FIGS. 3A and 3B). Cells that were double-labeled for BrdU and PLP constituted fewer than 10% of cells in both groups: 5.3±1.6% (Mean ±SEM) of all BrdU cells in the control group and 8.2±0.8% of all BrdU cells in the ECS group. There was a trend for a statistically significant difference between these ratios (Fisher exact test, p<0.07). By contrast, BrdU+NG2 double-labeled cells were observed commonly in both groups. These cells constituted 28.4±2.0% of all BrdU cells in the control group and 32.0±1.6% of all BrdU cells in the ECS group. The difference between these ratios was statistically significant (Fisher exact test, p<0.01). High quality staining was obtained with BrdU+RECA double-labeling and BrdU+NeuN+S100β triple-labeling. However, no BrdU+RECA, BrdU+NeuN, or BrdU+S100β double-labeled cells were seen in any of these studies (FIG. 3C).

SPRY2 Expression

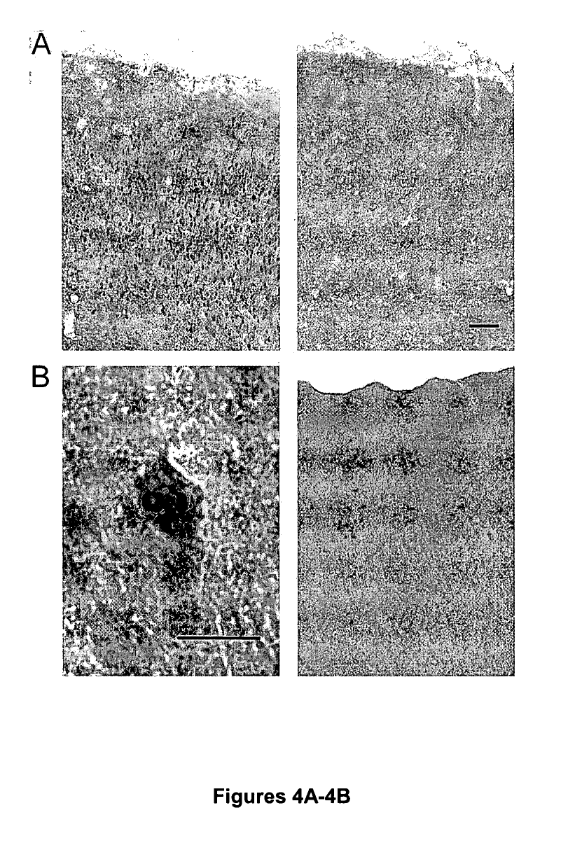

SPRY2-labeled cells were visible as evenly stained cell bodies throughout the brain, including the ILPFC (FIG. 4A). SPRY2-labeled cell bodies were occasionally organized in branching structures reminiscent of blood vessels, but this was seen in a patchy pattern and not evenly distributed (not shown). There was negligible nonspecific staining when the primary antibody was omitted from the staining procedure (FIG. 4B). ECS caused a five to six-fold decrease in SPRY2-labeled cells within the ILPFC (F(1,10)=4.403, p<0.05) (FIG. 1C).

Sprouty Expression in the Hippocampus (HIP)

In humans, the HIP has been implicated in the pathophysiology of depressive disorders (Sheline et al., Proc. Natl. Acad. Sci. USA 93: 3908-3913, 1996). In laboratory animals, antidepressant drugs and electroconvulsive seizure (ECS) promote HIP neurogenesis (Dranovsky et al., Biol. Psychiatry 12: 1136-1143, 2006); Warner-Schmidt et al., Hippocampus 16: 239-249, 2006) Recent work indicates that HIP neurogenesis is necessary for the therapeutic effects of antidepressants (Santarelli et al., Science 301: 805-809, 2003).

We have now shown that, in addition to decreasing expression in the ILPFC, rats subjected to ECS treatment also show decreased SPRY2 expression in the HIP. In addition, a corresponding increase cell proliferation and survival is also observed. Our results further indicate that the proliferating cells which survive 28 days adopt a neuronal fate. Finally, we have observed that treatment with chemical antidepressants decreases SPRY2 expression in the HIP, but to a lesser extent than observed with ECS.

Sprouty Function in Adult Mammalian Nervous System

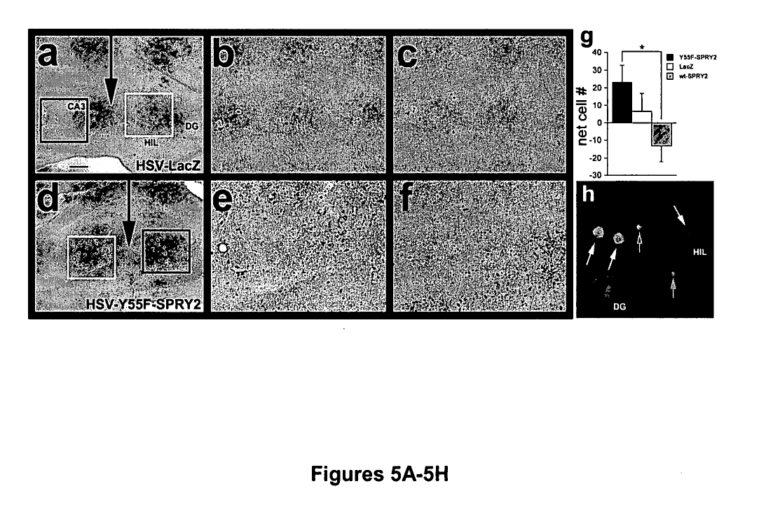

To determine if a decrease in SPRY2 expression was responsible for the increase in the observed ECS-induced cell proliferation, dominant negative SPRY2 (described in detail below; SEQ ID NO:6) was expressed in adult rat brains. This was accomplished by generating a herpes simplex virus (HSV) vector that encodes a dominant negative (Y55F) form of SPRY (HSV-Y55F-SPRY2). This vector was injected into the HIP, and seven days later an injection of bromodeoxyuridine (BrdU), which labels actively dividing cells, was given. The injected rat brain was examined on the following day; results are shown in FIGS. 5D-5F. These results indicate that expression of Y55F-SPRY in the HIP alone causes an increase in cellular proliferation (FIGS. 5D-5F) in the absence of ECS. To confirm that these results were not due to viral vector treatment or due to increases in expression of an arbitrary transgene, an HSV-based construct (HSV-LacZ) expressing β-galactosidase was injected into the contralateral HIP of the rat. The HSV-LacZ injection had negligible effects on cell proliferation (FIGS. 5A-5C). Thus, specific disruption of Sprouty function in an adult brain causes stimulation of cellular proliferation.

We further determined that the effects of HSV-Y55F-SPRY2 treatment results in greater numbers of BrdU cells 28 days following gene transfer, as compared to hemispheres transfected with LacZ transfected cells or transfected with wt-SPRY2 (FIG. 5G). Some of these BrdU cells show neuronal markers (FIG. 5H). Thus, by blocking Sprouty, we have enhanced cellular proliferation and at least some of these proliferating cells become neurons.

The identification of Sprouty as playing a pivotal role in inhibition of cellular proliferation provides a means for both treatment of psychiatric disorders, drug abuse or addiction, neurodegenerative diseases, and head trauma where either a decrease in cellular proliferation or an increase in cell death has been implicated, as well as a target for identification of therapeutics useful in promoting neurogenesis (e.g., in the treatment of such diseases).

Sprouty and Stress

Stress decreases HIP neurogenesis (Dranovsky et al., Biol. Psychiatry 12: 1136-1143, 2006; Warner-Schmidt et al., Hippocampus 16: 239-249, 2006; Banasr M, Valentine G W, Li X Y, Gourley S L, Taylor J R, Duman R S, “Chronic Unpredictable Stress Decreases Cell Proliferation in the Cerebral Cortex of the Adult Rat.” Biol. Psychiatry, in press, 2007), suggesting a role of stress in the development of depression. We believe stressors that decrease HIP neurogenesis also increase SPRY2 expression. This correlation can be determined using a restraint stress regimen in rats known to decrease HIP neurogenesis and single- and double-labeling immunohistochemistry. Rats can be exposed to either acute (1 hr) or chronic (1 hr/day for 10 days) stress and then examined for HIP SPRY2 expression 24 hr later. Alternatively, rats can be exposed to the same stressors, can be administered bromodeoxyuridine (BrdU) to label dividing cells, and can be examined for neurogenesis and SPRY2 expression 28 days later. These measurements can be performed with single- and double-labeling (BrdU and NeuN) immunohistochemistry and validated cell counting procedures (described herein) or using protein (western) immunoblotting for SPRY2 and PCNA (described herein and in Pliakas et al., J. Neurosci. 21: 7397-7403, 2001) in parallel studies. The latter approach is suitable when the baseline SPRY2 labeling is too high for immunohistochemistry. We believe these results can provide further evidence of SPRY2 involvement in suppression of neurogenesis.

Sprouty and Chemical Antidepressants

We have also shown that chronic treatment with chemical antidepressants decreases HIP SPRY2 expression, but the effects are not as pronounced as with ECS. This difference can explain the relative clinical efficacies of the two treatments. We therefore believe that inhibition or disruption of Sprouty can increase chemical antidepressant efficacy. BrdU labeling can be used to determine if direct (e.g., viral vector-mediated) alterations in HIP SPRY2 function affect the ability of a standard antidepressant (fluoxetine; FLX) to trigger neurogenesis (Santarelli et al., Science 301: 805-809, 2003). We believe disrupted SPRY2 function (by expression of Y55F-SPRY2) increases FLX-induced HIP neurogenesis, whereas elevated SPRY2 function (by expression of wild-type-SPRY2) decreases it.

Methods and Materials

The following methods and materials were used to perform the methods described herein.

Rats, ECS, and BrdU Procedures

A total of 10 Male Sprague-Dawley rats (Charles River Labs, Massachusetts) weighing 150-200 g at the beginning of the study were used. The rats were housed in groups of 3-4 under standard conditions with free access to food and water. ECS-treated rats (n=5) were administered seizures once daily for 10 days in the mornings by passing a 99-mA, 0.5 sec, 100 Hz current via earclip electrodes, using a current generator (Ugo Basile, Comerio, Italy). These parameters were chosen because they consistently elicit generalized seizures in all rats for treatment periods of this length (Öngür and Carlezon, unpublished observations). Control (sham)-treated rats (n=5) received similar handling including attachment of ear clips but no current was passed. To label proliferating cells, BrdU (Sigma, St. Louis, Mo.) was administered at 50 mg/kg in a 10 mg/ml solution by intraperitoneal (IP) injections twice daily for the same 10 day duration as ECS administration. One BrdU injection was given 30 min following ECS administration, and the second injection was given 12 hours later. Following the last day of ECS and BrdU administration, the rats were maintained for an additional 4 weeks with no interventions.

Tissue Processing

Rats were injected with sodium pentobarbital (130 mg/kg, IP), and perfused transcardially with phosphate-buffered saline (PBS) followed by 4% paraformaldehyde and overnight postfixation. The brains were equilibrated in 30% sucrose, cut into coronal sections (40 μm) on a microtome, and kept at 4° C. until staining. Six series of sections were cut for each brain, and adjacent full series through the forebrain were processed for each stain.

Staining Procedures

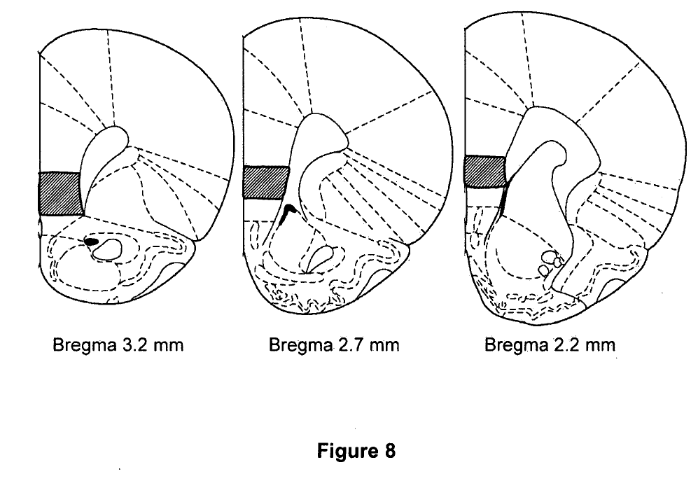

Sections were stained for bilateral cell counting (see below). Nissl staining was performed using standard procedures (Öngür et al., Proc. Natl. Acad. Sci. USA 95:13290-13295, 1998). Likewise, standard immunohistochemistry procedures (Carlezon et al., Science 277:812-814, 1997) were used to label BrdU-, PCNA-, PLP, NG2-, S100β-, NeuN-, RECA-, and SPRY2-immunoreactive cells (Table 2) within the ILPFC (FIG. 8) (Paxinos et al., The Rat Brain in Stereotaxic Coordinates, third ed., San Diego, Calif.: Academic Press, 1997). Staining was performed on mounted sections, except for the double-labeling, triple-labeling (see below), and SPRY2 studies, in which free-floating sections were used. For the studies using DAB staining, sections were first washed in PBS 3×10 min prior to incubation in 0.3% H₂O₂ for 30 min. They were then washed in PBS 3×10 min and placed in blocking solution (PBS with 5% normal serum and 0.3% Triton-X) for 30 min, followed by incubation in primary antibody in blocking solution overnight at room temperature. The next day, sections were washed in PBS 3×10 min and incubated in biotinylated secondary antibody (Vector Laboratories, Burlingame, Calif.) for 60 min. Following another PBS wash 3×10 min, sections were processed in the Vectastain ABC solution for 60 min. They were then washed in PBS 3×5 min and reacted in 0.05% diaminobenzidine (DAB) and 0.3% H₂O₂ for 4-8 min, producing a brown precipitate. Sections were then dried overnight, dehydrated in alcohol series and xylene and coverslipped.

TABLE 2

| | | | | |
|---|---|---|---|---|
| | Blocking | | | |
| | Normal | | | |
| Antigen | Serum | Primary Antibody | Antibody Source | Secondary Antibody |

DAB-based Staining

| | | | | |
|---|---|---|---|---|
| BrdU | Horse | Mouse monoclonal; 1:1000 | Chemicon (Temecula, Calif.) | Biotin-horse-anti-mouse |
| PCNA | Horse | Mouse monoclonal; 1:4000 | Chemicon (Temecula, Calif.) | Biotin-horse-anti-mouse |
| NG2a | Goat | Rabbit polyclonal; 1:250 | Chemicon (Temecula, Calif.) | Biotin-goat-anti-rabbit |
| PLPa | Goat | Rat monoclonal; 1:2000 | Immuno Diagnostics (Woburn, Mass) | Biotin-goat-anti-rat |
| SPRY2 | Goat | Rabbit polyclonal; 1:100 | Courtesy of Tarun Patel | Biotin-goat-anti-rabbit |

Immunofluorescence Staining

| | | | | |
|---|---|---|---|---|
| BrdU | Goat | Rat monoclonal; 1:100 | Accurate Chemical (Westbury, NY) | Alexa Fluor 633 (Molecular |
| | | | | Probes, Eugene, Ore.) |
| S100_b | Goat | Rabbit polyclonal; 1:500 | DakoCytomation (Glostrup, | Alexa Fluor 633 (Molecular |
| | | | Denmark) | Probes, Eugene, Ore.) |
| RECAb | Goat | Mouse monoclonal; 1:500 | Serotec (Oxford, UK) | Alexa Fluor 488 (Molecular |
| | | | | Probes, Eugene, Ore.) |
| NeuNb | Goat | Mouse monoclonal; 1:100 | Chemicon (Temecula, Calif.) | Alexa Fluor 488 (Molecular |
| | | | | Probes, Eugene, Ore.) |

DAB, diaminobenzidine;
BrdU, bromodeoxyuridine;
PCNA, proliferating cell nuclear antigen;
NG2, chondroitin-sulfate proteoglycan;
PLP, proteolipid protein;
SPRY2, Sprouty2;
S100_, beta-subunit of the S100 protein;
RECA, rat endothelial cell antigen;
NeuN, neuronal nuclear protein.
aStaining for NG2 and PLP was only carried out in conjunction with BrdU staining.
bStaining for S100_, RECA, and NeuN were only carried out in conjunction with BrdU staining.

Sections processed for BrdU staining were pretreated to enhance stain quality. They were incubated in 0.01 M citric acid, pH 6.0 at 95° C. for 10 min. Following a brief PBS wash, they were placed in a solution of 0.1% Trypsin and 0.1% CaCl₂ in 0.1M Tris buffer, pH 7.4 for 10 min. Following another brief PBS wash, they were incubated in 1N HCl for 30 min and then processed using the procedures described above.

Staining for PLP, NG2, S100β, RECA, and NeuN was only carried out in the context of double-labeling and triple-labeling with BrdU. For BrdU+PLP and BrdU+NG2 double-labeling, free floating sections were first stained for PLP or NG2 using the procedures described above, except the ImmunoPure Metal Enhanced DAB Kit (Pierce, Rockford, Ill.), which contains nickel and cobalt and produces a black precipitate, was used. Following completion of the PLP or NG2 staining protocol, sections were washed in PBS and transferred to the BrdU protocol, and then viewed with the normal (nonenhanced) DAB visualization procedure. In these sections, PLP or NG2 labeling produced dark profiles of neurites extending from lightly stained cell bodies, whereas BrdU labeling produced uniformly brown stained profiles in the size and shape of nuclei. Following the completion of staining, these sections were mounted on glass slides, dried, dehydrated, and coverslipped.

For BrdU+S100β+NeuN triple-labeling studies, and BrdU+RECA double-labeling studies, immunofluorescence staining was used. Sections were washed in potassium PBS (KPBS) and then incubated in 2N HCl at 37° C. for 30 min. After washing twice in KPBS, sections were incubated in blocking solution (KPBS with 3% normal goat serum and 0.3% Triton X-100) for 1 hour. Sections were then incubated for 3 days at 4° C. in blocking solution with rat anti-BrdU and either mouse anti-NeuN and rabbit anti-S100β or mouse anti-RECA antibody. Following washes in KPBS, fluorescent-labeled secondary antibodies (Alexa Fluor 488, 555, or 633) were added at a concentration of 1:200 in blocking solution for 1 hour at room temperature. Following the completion of staining, the sections were mounted, dried, dehydrated, and coverslipped.

Quantitative Measurements

Quantifying cell numbers and volume was performed blindly, so that the investigator was not aware of the treatment conditions. Left and right ILPFC were quantified separately. The ILPFC is defined by cytoarchitectonic criteria (Krettek et al., J. Comparative Neurol. 171:157-191, 1977) and is homologous to Brodmann's Area (BA) 25 in primates. The boundaries of this area were drawn on Nissl stained sections (every sixth section) and adjacent sections in which immunoreactive cells were labeled. The ILPFC extended 5-7 sections in each series. In Nissl stained sections, neurons were identified by the presence of large nuclei, heterogeneous chromatin and/or nucleoli, stained cytoplasm, and nonspherical shapes. Glial cells were small and round, with no stained cytoplasm.

All measurements on DAB-stained material were made using a brightfield microscope (Zeiss Axioskop2, Germany) and StereoInvestigator software (MicroBrightField, Williston, Vt.). ILPFC volume was estimated using planimetry: ILPFC area from all Nissl stained sections was summed and this number was multiplied by the distance between sections (240 μm). Cell density and numbers were estimated by using stereological methods (Gundersen et al., APMIS 96:857-881, 1988). Although sections were cut at 40 μm, after tissue processing and dehydration the final section thickness was approximately 7 μm. The number of counting windows sampled varied between 15 and 25 and the size of the counting window varied between 50×50 μm and 200×200 μm depending on the stain, in order to achieve a total cell count of more than 100, necessary to achieve reliable estimates using stereology (Gundersen et al., APMIS 96:857-881, 1988). In all, 159±7 neurons, 244±7 glia, 76±11 BrdU-immunoreactive cells, 109±17 PCNA-immunoreactive cells, and 125±31 SPRY2-immunoreactive cells were counted per hemisphere (mean ±SEM).

The total number of each cell type counted was divided by the volume sampled (number of windows×shrinkage factor×window depth×window area) to arrive at the estimated cell density for ILPFC. The shrinkage factor is the final section thickness estimated under the microscope divided by the section thickness at cutting (7 μm/40 μm). The volume of the ILPFC was previously calculated based on the area of ILPFC outlines in each section and cell density was multiplied by this volume to give the total number of cells in the ILPFC.

Sections from the mid-level of the ILPFC were selected in each brain for the analysis of double- and triple-labeling studies with BrdU and PLP, NG2, S100β, RECA, and NeuN. For PLP and NG2 double-labeling, all BrdU-immunoreactive cells in the ILPFC in these sections were counted and the number of double-labeled cells was recorded, yielding an estimate percentage of BrdU-immunoreactive cells that are also double-labeled for PLP and NG2. Between 32 and 155 BrdU-immunoreactive cells per brain were evaluated in each of these double-labeling studies. DABstained material was examined using a brightfield microscope. For BrdU-RECA and BrdU-S100β-NeuN experiments, a confocal microscope (Leica Microsystems; Exton, Pa.) was used to examine at least 30 BrdU-positive cells per animal. Colocalization of BrdU+ cells with the phenotypic markers was analyzed with Z-plane sectioning (1.5-μm steps).

Statistical Procedures

ILPFC volume, as well as neuron, glia, BrdU-, PCNA-, and SPRY2-immunoreactive cell density and number in this area were entered into a 2 (group)×11 (measure) ANOVA with hemisphere as a covariate. This analysis indicated statistically significant effects and was therefore followed by between-group effects testing by ANOVA for each 11 measures. The percentage of BrdU-immunoreactive cells that were double-labeled with other markers was analyzed separately as this measure was obtained without the use of unbiased estimation methods. To examine differences between two percentages, we used a two-tailed Fisher exact test.

Promoting Neurogenesis in a Subject

Based on the observation that Sprouty inhibits neurogenesis in the adult brain, the invention features methods for promoting neurogenesis in a subject, for example, a subject with a psychiatric disorder (e.g., depression, bipolar disorder, or post traumatic stress disorder), drug abuse or addiction (e.g., involving a drug described herein), a neurodegenerative disease (e.g., AD, PD, ALS, MS, FTD, HD, or prion disease), or a head trauma (e.g., stroke or physical injury), by administration of a composition that selectively inhibits Sprouty or binds to a Sprouty binding site (e.g., on a Sprouty interacting protein described herein) to a degree sufficient to promote neurogenesis. The compounds used in the treatment of methods of the invention may be, for example, compounds identified using a screening method described herein.

Dominant Negative Sprouty

Promoting neurogenesis in a subject (e.g., a subject with a psychiatric disorder, drug abuse or addiction, a neurodegenerative disease, or head trauma) can be accomplished using a dominant negative form of Sprouty. Dominant negative Sprouty proteins interfere with the activity of wild-type Sprouty (e.g., binding to GRB2, RAF, c-CBL, GAP1, or any interaction partner described herein, or membrane binding), thereby inhibiting the repression of neurogenesis caused by wild-type Sprouty.

In one example, introduction of a tyrosine-to-phenylalanine point mutation at position 55 of Sprouty subtype 2 (Y55F SPRY; SEQ ID NO:6) prevents activation (i.e., phosphorylation) of the mutant SPRY (Hanafusa et al., Nat. Cell Biol. 4:850-858, 2002; Kim et al. Nat. Rev. Mol. Cell. Biol. 5:441-450, 2004). Y55F SPRY competes for the same binding sites as wild-type, endogenous SPRY, but, lacking phosphorylation at position 55, cannot activate the processes (e.g., GRB2 binding) that result in inhibition of ERK/MAPK signaling. In a particular example, a corresponding mutation is generated in human Sprouty 2 (SEQ ID NO:7). Other mutations, including a tyrosine-to-alanine mutation (Y55A), can also be introduced at position 55 of Sprouty2 to generate a dominant negative Sprouty protein.

Additional dominant negative Sprouty proteins (e.g., dominant negative forms of Sprouty1, Sprouty2, Sprouty3, or Sprouty4) for use in the methods of the invention can be identified by testing an altered Sprouty protein such as a mutant Sprouty protein (e.g., a Sprouty protein with an insertion, a deletion, or a point mutation) or a Sprouty protein chemically modified for its ability to induce ERK/MAPK activation or neuronal differentiation in cell culture upon exogenous administration of the altered protein or expression of a vector coding for the altered protein. In one example, any tyrosine residue (e.g., position 53 of human Sprouty1, position 27 of human Sprouty3, or position 52 of human Sprouty4) in a Sprouty protein may be modified (e.g., to a phenylalanine or an alanine) to generate a candidate dominant negative Sprouty. Dominant negative proteins useful in the invention may include fragments of Sprouty (e.g., fragments including the conserved amino-terminal domain) and modified Sprouty fragments (e.g., containing a tyrosine to phenyalanine or tyrosine to alanine mutation). Any dominant negative proteins identified or a polynucleotide encoding a dominant negative protein can be administered to a patient in order to promote neurogenesis, thereby making them useful in treating a psychiatric disorder (e.g., depression, bipolar disorder, or post traumatic stress disorder), drug abuse or addiction (e.g., involving a drug described herein), a neurodegenerative disease (e.g., AD, PD, ALS, MS, FTP, HD, or prion disease), or a head trauma (e.g., stroke or physical injury).

Antibodies

A Sprouty antibody or an antibody that binds to a Sprouty binding site can also be used in the methods of the invention to promote neurogenesis. Antibodies to mammalian SPRY are commercially available (e.g., from U.S. Biological, Swampscott, Mass. or Upstate Group LLC, Charlottesville, Va.). Alternatively, antibodies to Sprouty or to a protein that competitively inhibits Sprouty binding at a Sprouty binding site (e.g., a monoclonal or polyclonal antibody) can be generated using methods standard in the art. These antibodies can be modified in any way to make them more appropriate for human administration. For example, they can be single-chain antibodies or humanized antibodies. These antibodies are administered by any route, formulation, frequency, or in any dose that achieves in vivo concentrations sufficient for increased neurogenesis (e.g., in the treatment of a psychiatric disorder, drug abuse or addiction, a neurodegenerative disease, or head trauma).

Gene Therapy

Decreases in Sprouty expression or activity to promote neurogenesis (e.g., to treat a psychiatric disorder, drug abuse or addiction, a neurodegenerative disease, or head trauma) can also be achieved through introduction of a gene vector into a subject. Any standard gene therapy vector and methodology can be employed for such administration.

To decrease expression of Sprouty for promoting neurogenesis (e.g., treating a psychiatric disorder, drug abuse or addiction, a neurodegenerative disease, or head trauma), RNA interference (RNAi) can be employed. Vectors containing a target sequence, such as a short (for example, 19 base pair) sense target sequence and corresponding antisense target sequence joined by a short (for example, 9 base pair) sequence capable of forming a stem-loop structure, of the Sprouty mRNA transcript can be administered to a subject (e.g., a subject with a psychiatric disorder, drug abuse or addiction, a neurodegenerative disease, or head trauma) to promote neurogenesis. When this vector is expressed in cells, small, inhibitory RNA (siRNA) molecules are generated from this stem-loop structure, and these bind to Sprouty mRNA transcripts, which results in increased degradation of the targeted mRNA transcripts relative to untargeted transcripts. To test the efficacy of different sequences in mammalian cell culture systems, the pSuper RNAi System (OligoEngine, Seattle, Wash.), for example, can be employed.

In another embodiment, reduction of Sprouty activity can be achieved through the administration to a subject a vector containing a gene coding for a dominant negative Sprouty protein such as human Y55F Sprouty protein (SEQ ID NO:7) as described herein to promote neurogenesis (e.g., to treat a psychiatric disorder, drug abuse or addiction, a neurodegenerative disease, or head trauma). Expression of this protein in the subject will reduce endogenous Sprouty activity and promote neurogenesis.

Small Molecule Sprouty Inhibitors

Small molecules (e.g., molecules with a molecular weight less than 3000, 2500, 2000, 1500, 1000, 750, or 500 Da) may be used in the methods of the invention to promote neurogenesis in a subject. Any small molecule that selectively inhibits Sprouty activity may be used in the treatment methods of the invention. In one embodiment, a small molecule identified using a screening method described herein is used in the methods of the invention (e.g., to treat a psychiatric disorder, drug abuse or addiction, a neurodegenerative disease, or head trauma). Such small molecules may be incorporated into pro-drugs (i.e., a compound that is metabolized to generate the active drug). Pro-drugs are described in detail in Higuchi and Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987 and Judkins et al., Synth. Commun. 26(23):4351-4367, 1996.

Combinations of Antidepressant Treatment and a Composition that Promotes Neurogenesis

As we have determined that chronic treatment with ECS or chemical antidepressants results in decreased SPRY2 expression, we believe that administration of a composition that selectively inhibits Sprouty or binds to Sprouty binding site (e.g., any of those described herein) can increase the efficacy of ECS-like treatments (e.g., electroconvulsive therapy (ECT) or chemical antidepressants in treatment of psychiatric disorders such as depression, bipolar disorder, and post traumatic stress disorder. Antidepressants include selective serotonin reuptake inhibitors (SSRIs) (e.g., citalopram, escitalopram oxalate, fluvoxamine, paroxetine, fluoxetine, and sertraline), monoamine oxidase inhibitors (MAOIs) (e.g., phenelzine, tranylcypromine), tricyclics (e.g., doxepin, clomipramine, amitriptyline, amitriptyline, maprotiline, desipramine, nortryptyline, desipramine, doxepin, trimipramine, imipramine, and protriptyline). Other antidepressants include buspirone, duloxetine, trazodone, venlafaxine, reboxetine, mirtazapine, nefazodone, and bupropion. In addition, antidepressant effects have also been associated with the use of mood stabilizing agents (e.g., lithium, valproic acid) (Calabrese et al. J. Clin. Psychpharmacol. 12: 53S-56S, 1992; Granneman et al., J. Clin. Psychiatry 57: 204-206, 1996; Guzzetta et al., J. Clin. Psychiatry. 68: 380-383, 2007), and recent studies suggest that kappa antagonists (e.g., nor-binaltorphimine, JDTic; Pliakas et al., J. Neurosci. 21: 7397-7403, 2001; Mague et al., J. Pharmacol. Exper. Ther. 305: 323-330, 2003), vasopressin (V1b) antagonists (e.g., SSR149415; Hodgson et al., Pharmacol. Biochem. and Behav. 86: 431-440, 2007) and corticotropin releasing factor (CRF) antagonists (e.g., CP-154,526 and R121919; Hodgson et al., Pharmacol Biochem and Behav 86: 431-440, 2007; Skelton et al., Psychopharmacol., in press) have antidepressant effects. Antidepressant effects are also associated with administration of dopamine D1-type receptor antagonists (Meloni et al., J. Neurosci. 26: 3855-3863, 2006). Exemplary selective D1 antagonists include ecopipam (SCH-39166), SCH-23390, SCH-23982, A-69024, SCH-12679, SKF-83566, ADX10061, and LE 300. Exemplary nonselective D1 antagonists include thioridazine, thiothixine, trifluoperazine, trifluperidol, bulbocapnine, (+)-butaclamol, fluphenazine, flupenthixol, fluspirilene, and haloperidol. Compounds with antidepressant effects also include omega-3 fatty acids and nucleosides such as uridine and cytidine (Carlezon et al., Biol. Psychiatry 51: 882-889, 2002; Carlezon et al., Biol. Psychiatry 57: 243-250, 2005). Any of these agents can be used in the treatment methods of the invention.

The increase in efficacy can reduce the symptoms of depression to greater extent than with the chemical antidepressant or the antidepressant therapy alone. In other embodiments, administration of a selective inhibitor of Sprouty can reduce the dosing amounts, frequency of doses, or time over which the subject is treated using the antidepressant therapy or chemical antidepressant as compared to in the absence of the Sprouty inhibitor.

Formulation of Pharmaceutical Compositions

The administration of any compound described herein or identified using the screening methods of the invention can be by any suitable means that results in a concentration of the compound that promote neurogenesis (e.g., to treat a psychiatric disorder, drug abuse or addiction, a neurodegenerative disease, or head trauma). The compound can be contained in any appropriate amount in any suitable carrier substance, and is generally present in an amount of 1-95% by weight of the total weight of the composition. The composition can be provided in a dosage form that is suitable for the oral, parenteral (e.g., intravenously, intramuscularly, intracranially, intrathecally), rectal, cutaneous, nasal, vaginal, inhalant, skin (patch), ocular, or intracranial administration route. The pharmaceutical compositions can be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions can be formulated to release the active compound immediately upon administration or at any predetermined time or time period after administration. The latter types of compositions are generally known as controlled release formulations, which include (i) formulations that create a substantially constant concentration of a compound within the body over an extended period of time; (ii) formulations that after a predetermined lag time create a substantially constant concentration of a compound within the body over an extended period of time; (iii) formulations that sustain a compound's action during a predetermined time period by maintaining a relatively constant, effective level of the compound in the body with concomitant minimization of undesirable side effects associated with fluctuations in the plasma level of the compound (sawtooth kinetic pattern); (iv) formulations that localize action of a compound, e.g., spatial placement of a controlled release composition adjacent to or in the diseased tissue (e.g., within a particular region of the brain affected by the disease); (v) formulations that achieve convenience of dosing, e.g., administering the composition once per week or once every two weeks; and (vi) formulations that target the action of a compound by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

Parenteral Compositions

A compound described herein or identified using the methods of the invention or a composition containing the compound can be administered parenterally by injection, infusion, or implantation (subcutaneous, intravenous, intramuscular, intraperitoneal, intracranial, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Parenteral compositions used in the methods of the invention can be in a form suitable for sterile injection. To prepare such a composition, the compound is dissolved or suspended in a parenterally acceptable liquid vehicle. Among acceptable vehicles and solvents that can be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation can also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent can include 10-60% w/w of propylene glycol or the like.

Nervous System Administration

In many cases, it is desirable that decreased Sprouty expression or activity be limited to the nervous system, or even further limited to the tissues of the nervous system in which increased neurogenesis is desired (e.g., tissues particularly affected by the psychiatric disorder, drug abuse or addiction, the neurodegenerative disease, or head trauma being treated). In the case of administration of exogenous compounds such as Sprouty2 antibodies or dominant negative Sprouty2, delivery to the affected of the nervous system can be achieved, for example, by the methods outlined below.

Treatments that promote neurogenesis (e.g., treatments for a psychiatric disorder, drug abuse or addiction, a neurodegenerative disease, or head trauma) can be hampered by the inability of an active, therapeutic compound to cross the blood-brain barrier (BBB). Strategies to delivery of compounds of the invention to such disorders and diseases include strategies to bypass the BBB (e.g., intracranial administration via craniotomy and intrathecal administration), and strategies to cross the BBB (e.g., the use of compounds or treatments such as ECT) that increase permeability of the BBB in conjunction with systemic administration of therapeutic compositions, and modification of compounds to increase their permeability or transport across the blood-brain barrier.

Craniotomy, a procedure known in the art, can be used with for delivery of therapeutic compositions to the brain. In this approach, a opening in made in the subject's cranium, and a compound is delivered via a catheter. This approach can be used to target a compound to a specific area of the brain (e.g., the substantia nigra for treating Parkinson's disease or the cortex for treating Alzheimer's disease).

Intrathecal administration provides another means of bypassing the blood brain barrier for drug delivery. Briefly, drugs are administered to the spinal cord, for example, via lumbar puncture or through the use of devices such as pumps. Lumbar puncture is preferable for single or infrequent administration, whereas constant and/or chronic administration can be achieved using any commercially available pump attached to an intraspinal catheter, for example, a pump and catheter made by Medtronic (Minneapolis, Minn.).

To allow for delivery across the BBB, compositions of the invention can be administered along with a compound or compounds that induce a transient increase in permeability of the blood-brain barrier. Such compounds include mannitol, Cereport (RMP-7), and KB-R7943, a Na⁺/Ca⁺⁺ exchange blocker. In another embodiment, permeability of the blood-brain barrier can be increased using ECT (also known as electro-convulsive shocks) (Awasthi et al., Pharmacol. Res. Commun. 14:983-992, 1982) in conjunction with administration of a composition that promotes neurogenesis.

In another embodiments, compounds (e.g., compounds identified using screening methods of the invention) can be modified (e.g., lipidated, acetylated) to increase transport across the blood-brain barrier following systemic administration (e.g., parenteral), by using chemical modifications standard in the art. In one embodiment, compounds of the invention are conjugated to peptide vectors that are transported across the BBB. For example, compounds can be conjugated to a monoclonal antibody to the human insulin receptor as described by Partridge (Jpn. J. Pharmacol. 87:97-103, 2001), thus permitting the compound to be transported across the BBB following systemic administration. Compounds (e.g., those identified using screen methods described herein) can be conjugated to such peptide vectors, for example, using biotin-streptavidin technology. In the case of treatments using a gene therapy vector, in place of or in addition to localizing delivery of the vector, promoters that restrict expression to particular subpopulations of neurons can be employed. For example, expression of a gene therapy vector in treatment of PD can be limited to dopaminergic neurons through the use of a tyrosine hydroxylase promoter.

Dosages

The dosage of any compound described herein or identified using a method described herein depends on several factors, including: the administration method, the amount, rate, or extent of promotion of neurogenesis desired, the condition (e.g., psychiatric disorder, drug abuse or addiction, neurodegenerative disease, or head trauma) to be treated, the severity of the condition to be treated, whether the condition is to be treated or prevented, and the age, weight, and health of the subject to be treated. Also considered is whether the compound is being administered alone or in conjunction with another agent such as a chemical antidepressant or therapy (e.g., ECT).

With respect to the treatment methods of the invention, it is not intended that the administration of a compound to a subject be limited to a particular mode of administration, dosage, or frequency of dosing; the invention contemplates all modes of administration, including intracranial, intrathecal, intramuscular, intravenous, intraperitoneal, intravesicular, intraarticular, subcutaneous, or any other route sufficient to provide a dose adequate to promote neurogenesis (e.g., to treat a psychiatric disorder, drug abuse or addiction, a neurodegenerative disease, or head trauma). The compound can be administered to the subject in a single dose or in multiple doses. For example, a compound described herein or identified using screening methods of the invention can be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compound. For example, the dosage of a compound can be increased if the lower dose does not provide sufficient neurogenesis. Conversely, the dosage of the compound can be decreased after sufficient neurogenesis has been promoted or if the psychiatric disorder, drug abuse or addiction, neurodegenerative disease, or damage resulting from head trauma has been reduced or eliminated.

While the attending physician ultimately will decide the appropriate amount and dosage regimen, a therapeutically effective amount of a compound described herein (e.g., an antibody that specifically binds Sprouty, dominant negative Sprouty) or identified using the screening methods of the invention, can be, for example, in the range of 0.0035 μg to 1000 mg/kg body weight/day or 0.010 μg to 140 μg/kg body weight/week. Desirably a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount can be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of a compound can be, for example, in the range of 100 μg/m² to 100,000 μg/m² administered every other day, once weekly, or every other week. In a desirable embodiment, the therapeutically effective amount is in the range of 1000 μg/m² to 20,000 μg/m², for example, at least 1000, 1500, 4000, or 14,000 μg/m² of the compound administered daily, every other day, twice weekly, weekly, or every other week.

Screening Methods to Identify Candidate Therapeutic Compounds

The invention also features screening methods for the identification of compounds that bind to a Sprouty protein, a Sprouty fragment, or a Sprouty target protein (e.g., GRB2, c-CBL, RAF, or any of those described herein) or decrease expression of Sprouty and can therefore be used to promote neurogenesis (e.g., in the treatment of a psychiatric disorder, drug abuse or addiction, a neurodegenerative disease, or head trauma).

Screening Assays

Screening assays to identify compounds that bind to Sprouty, a Sprouty fragment, or a Sprouty target protein (e.g., GRB2, c-CBL, RAF, or any of those described herein) or decrease expression of Sprouty are carried out by standard methods. The screening methods can involve high-throughput techniques. In addition, these screening techniques can be carried out in cultured cells or in organisms such as worms, flies, or yeast. Screening in these organisms can include the use of polynucleotides homologous to human Sprouty proteins (e.g., Sprouty proteins from mouse, rat, or fly).

Any number of methods are available for carrying out such screening assays. According to one approach, candidate compounds are added at varying concentrations to the culture medium of cells expressing a polynucleotide coding for Sprouty. Gene expression is then measured, for example, by standard Northern blot analysis (Ausubel et al., Current Protocols in Molecular Biology, Wiley Interscience, New York, 1997), using any appropriate fragment prepared from the polynucleotide molecule as a hybridization probe. The level of gene expression in the presence of the candidate compound is compared to the level measured in a control culture medium lacking the candidate molecule. A compound which promotes a decrease in Sprouty expression is considered useful in the invention; such a molecule can be used, for example, as a therapeutic to promote neurogenesis, e.g., for treatment of a psychiatric disorder, drug abuse or addiction, a neurodegenerative disease, or head trauma (e.g., those described herein).

If desired, the effect of candidate compounds can, in the alternative, be measured against the level of polypeptide production using the same general approach and standard immunological techniques, such as western blotting or immunoprecipitation with an antibody specific for Sprouty. For example, immunoassays can be used to detect or monitor the expression of Sprouty. Polyclonal or monoclonal antibodies which are capable of binding to such a polypeptide can be used in any standard immunoassay format (e.g., ELISA, western blot, or RIA assay) to measure the level of Sprouty. A compound which promotes a decrease in the expression of Sprouty is considered particularly useful. Again, such a molecule can be used, for example, as a therapeutic to promote neurogenesis, for example, for treatment of a psychiatric disorder (e.g., depression, bipolar disorder, or post traumatic stress disorder), drug abuse or addiction (e.g., those described herein), a neurodegenerative disease (e.g., AD, PD, ALS, MS, FTD, HD, or prion disease), or a head trauma (e.g., stroke or physical injury).

Alternatively, or in addition, candidate compounds can be screened for those which specifically bind to Sprouty, a Sprouty fragment, or a Sprouty target protein (e.g., GRB2, c-CBL, RAF, or any of those described herein). The efficacy of such a candidate compound is dependent upon its ability to interact with the polypeptide. Such an interaction can be readily assayed using any number of standard binding techniques and functional assays (e.g., those described in Ausubel et al., supra). For example, a candidate compound can be tested in vitro for interaction with and binding to Sprouty.

In one particular embodiment, a candidate compound that binds to Sprouty, a Sprouty fragment, or a Sprouty target protein (e.g., GRB2, c-CBL, RAF, or any of those described herein) can be identified using a chromatography-based technique. For example, recombinant Sprouty can be purified using standard techniques from cells engineered to express Sprouty and can be immobilized on a column. A solution of candidate compounds is then passed through the column, and a compound specific for Sprouty is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach can also be used as therapeutics to promote neurogenesis, for example, to treat a psychiatric disorder (e.g., depression, bipolar disorder, or post traumatic stress disorder), drug abuse or addiction (e.g., those described herein), a neurodegenerative disease (e.g., AD, PD, ALS, MS, FTD, HD, or prion disease), or a head trauma (e.g., stroke or physical injury). Compounds which are identified as binding to Sprouty, a Sprouty fragment, or a Sprouty target protein (e.g., GRB2, c-CBL, RAF, or any of those described herein) with an affinity constant less than or equal to 10 mM are considered particularly useful in the invention. Compounds that bind to a Sprouty target protein may, in certain embodiments, be tested for their ability to decrease binding (e.g., specific binding) of Sprouty to the Sprouty target protein.

Potential candidate compounds include organic molecules, peptides, peptide mimetics, polypeptides, and antibodies that bind to Sprouty, or a polynucleotide encoding Sprouty and thereby decrease its activity (e.g., siRNA).

Polynucleotide sequences coding for Sprouty can also be used in the discovery and development of compounds to promote neurogenesis, for example, to treat a psychiatric disorder (e.g., depression, bipolar disorder, or post traumatic stress disorder), drug abuse or addiction (e.g., those described herein), a neurodegenerative disease (e.g., AD, PD, ALS, MS, FTD, HD, or prion disease), or a head trauma (e.g., stroke or physical injury). The polynucleotide sequences encoding the amino terminal regions of the encoded polypeptide or Shine-Delgamo or other translation facilitating sequences of the respective mRNA can be used to construct antisense sequences to control the expression of the coding sequence of interest. Polynucleotides encoding fragments of Sprouty may, for example, be expressed such that RNA interference takes place, thereby reducing expression of Sprouty and promoting neurogenesis.

Optionally, compounds identified in any of the above-described assays can be confirmed as useful in promoting neurogenesis (e.g., delaying or ameliorating a psychiatric disorder, drug abuse or addiction, a neurodegenerative disease, or head trauma in either standard tissue culture methods or animal models and, if successful, can be used as therapeutics for promoting neurogenesis, for example, for treating a psychiatric disorder (e.g., depression, bipolar disorder, or post traumatic stress disorder), drug abuse or addiction (e.g., those described herein), a neurodegenerative disease (e.g., AD, PD, ALS, MS, FTD, HD, or prion disease), or a head trauma (e.g., stroke or physical injury) in human subjects. Administration to the animal can be achieved using any of the administration known in the art such as those disclosed herein. Neurogenesis or treatment of symptoms of any disorder, disease, or condition (e.g., those described herein) may be measured using any method known in the art (e.g., increases in neurogenesis can be identified by measuring BrdU incorporation into dividing cells). A compound which increases neurogenesis in an animal, as compared to a control, is identified as a compound with therapeutic potential. Suitable controls include control animals not receiving the compound (e.g., animals receiving a sham administration or an inactive compound), or in the case of compounds administered directly to the brain, a compound may be administered to one brain hemisphere and neurogenesis may be compared between the hemisphere receiving the compound and the hemisphere not receiving the compound (e.g., receiving a sham administration or a control compound).

Small molecules, in particular, provide useful candidate therapeutics. In particular embodiments, such molecules have a molecular weight below 2,000 Da, can have a molecular weight between 300 and 1,000 Da or between 400 and 700 Da. These small molecules can be organic molecules.

Test Compounds and Extracts

In general, compounds capable of promoting neurogenesis, for example, for treating a psychiatric disorder (e.g., depression, bipolar disorder, or post traumatic stress disorder), drug abuse or addiction (e.g., those described herein), a neurodegenerative disease (e.g., AD, PD, ALS, MS, FTD, HD, or prion disease), or a head trauma (e.g., stroke or physical injury) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedures of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based (e.g., mircoRNA and siRNA) compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in promoting neurogenesis (e.g., for treating a psychiatric disorder, drug abuse or addiction, a neurodegenerative disease, or head trauma) should be employed whenever possible.

When a crude extract is found to have an activity that binds Sprouty, a Sprouty fragment, or Sprouty target protein (e.g., GRB2, c-CBL, RAF, or any of those described herein) or decreases Sprouty expression, further fractionation of the positive lead extract is necessary to isolate chemical constituents responsible for the observed effect. Thus, the goal of the extraction, fractionation, and purification process is the characterization and identification of a chemical entity within the crude extract having activity that can be useful in treating a psychiatric disorder (e.g., depression, bipolar disorder, or post traumatic stress disorder), drug abuse or addiction (e.g., involving a drug described herein), a neurodegenerative disease (e.g., AD, PD, ALS, MS, FTD, HD, or prion disease), or a head trauma (e.g., stroke or physical injury). Methods of fractionation and purification of such heterogeneous extracts are known in the art. If desired, compounds shown to be useful agents for the treatment of a psychiatric disorder (e.g., depression, bipolar disorder, or post traumatic stress disorder), drug abuse or addiction, a neurodegenerative disease (e.g., AD, PD, ALS, MS, FTD, HD, or prion disease), or a head trauma (e.g., stroke or physical injury) are chemically modified according to methods known in the art.

### Other Embodiments

All patents, patent applications, and publications mentioned in this specification are herein incorporated by reference to the same extent as if each independent patent, patent application, or publication was specifically and individually indicated to be incorporated by reference.

What is claimed is:

1. A method for promoting neurogenesis in a mammalian subject, said method comprising administering to said subject a composition that selectively inhibits Sprouty to a degree sufficient to promote neurogenesis.

2. The method of claim 1, wherein said composition comprises a compound that specifically binds Sprouty.

3. The method of claim 2, wherein said compound is an antibody that specifically binds Sprouty, or a Sprouty-binding fragment thereof.

4. The method of claim 1, wherein said composition comprises a dominant negative form of Sprouty.

5. The method of claim 4, wherein said dominant negative form of Sprouty comprises a Y55F mutation.

6. The method of claim 1, wherein said composition comprises an siRNA molecule that specifically binds to an mRNA encoding Sprouty.

7. The method of claim 1, wherein said composition comprises a vector encoding an siRNA that specifically binds to a mRNA encoding Sprouty.

8. The method of claim 1, wherein said composition comprises a vector encoding a dominant negative form of Sprouty.

9. The method of claim 8, wherein said dominant negative form of Sprouty comprises a Y55F mutation.

10. The method of claim 1, wherein said Sprouty is a human Sprouty.

11. The method of claim 1, wherein said subject has a psychiatric disorder and said promoting of neurogenesis treats said psychiatric disorder.

12. The method of claim 11, wherein said psychiatric disorder is depression, bipolar disorder, or post traumatic stress disorder.

13. The method of claim 11, wherein said method further comprises administration of a chemical antidepressant or antidepressant therapy to said subject.

14. The method of claim 1, wherein said subject has a neurodegenerative disease and said promoting of neurogenesis treats said neurodegenerative disease.

15. The method of claim 14, wherein said neurodegenerative disease is Alzheimer's disease, Parkinson's disease, amyotrophic lateral sclerosis, multiple sclerosis, frontotemporal dementia, Huntington's disease, or prion disease.

16. The method of claim 1, wherein said subject abuses or is addicted to a drug, and said promoting of neurogenesis decrease use of said drug or treats said addiction.

17. The method of claim 1, wherein said subject has suffered a head trauma, and said promoting of neurogenesis treats said trauma.

18. The method of claim 17, wherein said head trauma is stroke or physical injury.

19. The method of claim 1, wherein said subject is a human.

20. A method for promoting neurogenesis in a subject, said method comprising administering to said subject a composition that inhibits Sprouty activity by binding to a Sprouty binding site to a degree sufficient to promote neurogenesis.

21. The method of claim 20, wherein said composition comprises a dominant negative form of Sprouty.

22. The method of claim 20, wherein said Sprouty binding site is on GRB2 or c-CBL.

23. A method for identifying a candidate compound useful in promoting neurogenesis, said method comprising the steps:
(a) contacting a compound with a Sprouty protein; and
(b) measuring the binding of said compound to said Sprouty protein, wherein specific binding of said compound to said Sprouty protein identifies said compound as a candidate compound useful in promoting neurogenesis in a subject.

24. The method of claim 23, wherein said compound is selected from a chemical library.

25. The method of claim 23, wherein said Sprouty is a human Sprouty.

26. The method of claim 23, said method further comprising step:
(c) administering to a non-human mammal a compound identified in step (b) as specifically binding Sprouty, wherein a compound that increases neurogenesis in said mammal is a identified a potential therapeutic compound.

27. The method of claim 26, wherein said mammal has at least one symptom of a psychiatric disorder, neurodegenerative disease, or head trauma.

28. The method of claim 27, wherein said mammal has said psychiatric disorder or said neurodegenerative disease, or has suffered a head trauma.

29. A method for identifying a candidate compound useful in promoting neurogenesis in a subject, said method comprising the steps:
(a) contacting a compound with a cell or cell extract which comprises a polynucleotide encoding a Sprouty protein; and
(b) measuring the level of Sprouty expression in said cell or cell extract, wherein a decreased level of Sprouty expression in the presence of said compound relative to the level in the absence of said compound identifies said compound as a candidate compound useful in promoting neurogenesis in a subject.

30. The method of claim 29, wherein said compound is selected from a chemical library.

31. The method of claim 29, wherein said Sprouty is a human Sprouty.

32. The method of claim 29, said method further comprising step:
(c) administering to a non-human mammal a compound identified in step (b) as reducing Sprouty expression, wherein a compound that increases neurogenesis in said mammal is a identified a potential therapeutic compound.

33. The method of claim 32, wherein said mammal has at least one symptom of a psychiatric disorder, neurodegenerative disease, or head trauma.

34. The method of claim 33, wherein said mammal has said psychiatric disorder or said neurodegenerative disease, or a has suffered a head trauma.


SEQUENCE LISTING (7 sequences)

SEQ ID NO: 1 - protein, 319 aa, Homo sapiens
MDPQNQHGSGSSLVVIQQPSLDSRQRLDYEREIQPTAILSLDQIKAIRGSNEYTEGPSVVKRPAPRTAPRQEKHERTHEIIPINVNNNYEHRHTSHLGHAVLPSNARGPILSRSTSTGSAASSGSNSSASSEQGLLGRSPPTRPVPGHRSERAIRTQPKQLIVDDLKGSLKEDLTQHKFICEQCGKCKCGECTAPRTLPSCLACNRQCLCSAESMVEYGTCMCLVKGIFYHCSNDDEGDSYSDNPCSCSQSHCCSRYLCMGAMSLFLPCLLCYPPAKGCLKLCRRCYDWIHRPGCRCKNSNTVYCKLESCPSRGQGKPS

SEQ ID NO: 2 - protein, 315 aa, Homo sapiens
MEARAQSGNGSQPLLQTPRDGGRQRGEPDPRDALTQQVHVLSLDQIRAIRNTNEYTEGPTVVPRPGLKPAPRPSTQHKHERLHGLPEHRQPPRLQHSQVHSSARAPLSRSISTVSSGSRSSTRTSTSSSSSEQRLLGSSFSSGPVADGIIRVQPKSELKPGELKPLSKEDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT

SEQ ID NO: 3 - protein, 288 aa, Homo sapiens
MDAAVTDDFQQILPIEQLRSTHASNDYVERPPAPCKQALSSPSLIVQTHKSDWSLATMPTSLPRSLSQCHQLQPLPQHLSQSSIASSMSHSTTASDQRLLASITPSPSGQSIIRTQPGAGVHPKADGALKGEAEQSAGHPSEHLFICEECGRCKCVPCTAARPLPSCWLCNQRCLCSAESLLDYGTCLCCVKGLFYHCSTDDEDNCADEPCSCGPSSCFVRWAAMSLISLFLPCLCCYLPTRGCLHLCQQGYDSLRRPGCRCKRHTNTVCRKISSGSAPFPKAQEKSV

SEQ ID NO: 4 - protein, 299 aa, Homo sapiens
MEPPIPQSAPLTPNSVMVQPLLDSRMSHSRLQHPLTILPIDQVKTSHVENDYIDNPSLALTTGPKRTRGGAPELAPTPARCDQDVTHHWISFSGRPSSVSSSSSTSSDQRLLDHMAPPPVADQASPRAVRIQPKVVHCQPLDLKGPAVPPELDKHFLLCEACGKCKCKECASPRTLPSCWVCNQECLCSAQTLVNYGTCMCLVQGIFYHCTNEDDEGSCADHPCSCSRSNCCARWSFMGALSVVLPCLLCYLPATGCVKLAQRGYDRLRRPGCRCKHTNSVICKAASGDAKTSRPDKPF

SEQ ID NO: 5 - protein, 314 aa, Rattus norvegicus
MEARAQSGSGSQPLLQSPHDSGRQRGEPDPRDALTQQVHVLSLDQIRAIRNTNEYTEGPTVVPRPGLKPAPRPSTQHKHERLHGLPEHRQPPRLQPTQVHSSRAALSRSVSTVSSGSRSSTRTSTSSSSSEQRLLGSSFSPGPAVDGIIRVQPKSELKSGELKPLSRDDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT

SEQ ID NO: 6 - protein, 314 aa, Rattus norvegicus
MEARAQSGSGSQPLLQSPHDSGRQRGEPDPRDALTQQVHVLSLDQIRAIRNTNEFTEGPTVVPRPGLKPAPRPSTQHKHERLHGLPEHRQPPRLQPTQVHSSRAALSRSVSTVSSGSRSSTRTSTSSSSSEQRLLGSSFSPGPAVDGIIRVQPKSELKSGELKPLSRDDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT

SEQ ID NO: 7 - protein, 315 aa, Homo sapiens
MEARAQSGNGSQPLLQTPRDGGRQRGEPDPRDALTQQVHVLSLDQIRAIRNTNEFTEGPTVVPRPGLKPAPRPSTQHKHERLHGLPEHRQPPRLQHSQVHSSARAPLSRSISTVSSGSRSSTRTSTSSSSSEQRLLGSSFSSGPVADGIIRVQPKSELKPGELKPLSKEDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT